(19) United States
(12) Patent Application Publication
(10) Pub. No.: US 2016/0066986 A1
(43) Pub. Date: Mar. 10, 2016

(54) ELECTROSURGICAL INSTRUMENT FOR COAGULATING OR ABLATING BODY TISSUE

(71) Applicant: OLYMPUS WINTER & IBE GMBH, Hamburg (DE)

(72) Inventor: Hanno WINTER, Berlin (DE)

(21) Appl. No.: 14/442,657

(22) PCT Filed: Oct. 31, 2013
(86) PCT No.: PCT/EP13/72814

§ 371 Date: May 13, 2015

(30) Foreign Application Priority Data
Nov. 13, 2012 (DE) 10 2012 220 658.2

(51) Int. Cl. A61B 18/14

(52) U.S. Cl. CPC A61B 18/1487; A61B 18/149; A61B 18/14; A61B 2018/00642

(57) ABSTRACT

The invention relates to a bipolar electrosurgical instrument for ablating biological tissue, including an elongated shaft and two ablation electrodes, which are arranged one behind the other on the shaft in the longitudinal direction of the shaft and form a surface portion of the shaft, the ablation electrodes being electrically conductive and electrically separated from each other by an insulator, wherein the instrument has at least one measurement electrode, which is electrically insulated from the ablation electrodes and arranged between the two ablation electrodes in the immediate vicinity of one of the two ablation electrodes.

The invention relates to a bipolar electrosurgical instrument for ablating biological tissue, comprising an elongate shaft and comprising two ablation electrodes, which are arranged in succession on the shaft in the longitudinal direction of the shaft, form a surface portion of the shaft, are electrically conductive, and are electrically separated from one another by an insulator.

Electrosurgical instruments of the type set forth at the outset are known from the prior art and are used, for example, in endovenous treatment of venous insufficiency. For the treatment, the instrument is introduced into a vein and slowly withdrawn from proximal to distal under the emission of radiofrequency (RF) currents (approximately 0.2 MHz to 3 MHz), which are generated by e.g. a generator, as a result of which the vein is thermally obliterated.

Observations have been made that a blood clot may adhere to the electrodes or the insulator during the ablation of blood vessels, as a result of which the treatment must frequently be interrupted—for the purposes of removing the adhering blood clot.

It is therefore an object of the present invention to provide an improved electrosurgical instrument, by means of which treatment interruptions can be reduced.

In the electrosurgical instrument of the type set forth at the outset, this object is achieved by virtue of the instrument having at least one measurement electrode, which is electrically insulated from the ablation electrodes and arranged between the two ablation electrodes in the immediate surroundings of one of the two ablation electrodes.

Advantageously, this allows a measurement current for measuring the electrical resistance between the ablation electrode and the measurement electrode associated with this ablation electrode to be applied between a first ablation electrode and a first measurement electrode associated with this ablation electrode. As soon as a clot is formed at the edges of the electrode during an RF treatment by way of the ablation electrode, this can be detected by means of the measurement electrode by way of an increase in resistance. This applies accordingly to the second ablation electrode and a second measurement electrode associated with this ablation electrode.

This invention includes the discovery that the degree of adhesion of the blood clot at the electrodes or at the insulator situated between the electrodes is decisively determined by the emitted temperature dose. Since the highest power density always sets in along the current paths with the lowest resistance, these regions receive a comparatively high temperature dose during a normal application, as a result of which adhesion between the blood clot and the electrodes is caused.

The invention also includes the discovery that it is also not possible to prevent this effect by controlling the output power by way of the tissue resistance since summed electrical resistance measurable between the two ablation electrodes is not sensitive enough to be able to react to critical changes in resistance of small, compared to the overall volume through which current flows, volume regions.

Compared to conventional measurement of the summed resistance via the ablation electrodes, a clot can be detected much earlier by embodying two measurement electrodes between the insulator and ablation electrodes. The power of the generator can be reduced more quickly, as a result of which local overheating of the electrode edges is prevented. Accordingly, adhesion of blood or tissue at the electrode edges and unwanted treatment interruptions are reduced.

Within the scope of the present invention, an ablation electrode is to be understood to mean an electrically conductive electrode which is suitable for emitting an ablation current and/or a coagulation current. Therefore, the term ablation electrodes expressly also comprises coagulation electrodes or similar electrodes.

In order to be able to detect a blood clot as early as possible, a measurement electrode is preferably arranged in the direct vicinity of an ablation electrode. Preferably, a first measurement electrodes is arranged in the direct vicinity of a first ablation electrode. The second measurement electrode can be arranged in the direct vicinity of a second ablation electrode. Here, in particular, the direct vicinity should be understood to mean that no further electrode is situated between an ablation electrode and the measurement electrode associated therewith.

Advantageously, the measurement electrode is separated from the ablation electrode by a second, narrow insulator. It was also found to be advantageous if the distance between a respective ablation electrode and the immediately adjacent measurement electrode is substantially smaller than the distance between the measurement electrodes themselves. Thus, the distance between the measurement electrodes is preferably at least five times greater than the distance between a respective measurement electrode and the immediately adjacent ablation electrode.

In order to ensure a particularly uniform current emission, the ablation electrode and/or the measurement electrode can each have a ring-shaped embodiment. It was found to be advantageous if the measurement electrodes are arranged coaxially with respect to the shaft. A ring-shaped embodiment does not necessarily mean that the electrodes enclose the shaft continuously in this case. Thus, a measurement electrode can, for example, also be formed by a number of very small area, e.g. approximately punctiform, electrodes, which are arranged around the shaft in a ring-shaped manner.

In relation to the longitudinal direction of the shaft, the ablation electrodes can each have a substantially identical cross-sectional measure.

In order to be able to embody the instrument with a particularly compact design, the surface of a measurement electrode can be smaller in terms of area than the surface of an ablation electrode. Preferably, the surface of an ablation electrode is at least ten times larger than the surface of the associated measurement electrode. Preferably the width, i.e. the extent in the longitudinal direction of the shank, of the respective ring electrodes forming the measurement electrode is less than a quarter of the diameter thereof.

The shaft and/or the electrodes of the electrosurgical instrument can have a flexible embodiment, at least in sections, as a result of which the instrument is suitable for venous ablation in particular. The shaft and/or the electrodes of the electrosurgical instrument can also be rigid, promoting the use of the instrument for interstitial ablation, e.g. tumor treatment.

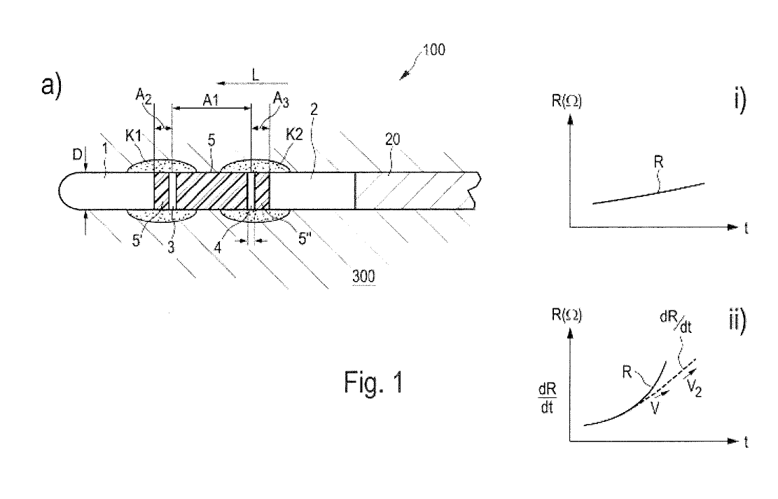

The invention also leads to a method for operating an electrosurgical instrument comprising the following steps:
applying a bipolar RF voltage to the ablation electrodes,
determining an electrical resistance and/or an increase in electrical resistance between an ablation electrode and a measurement electrode,
reducing the bipolar RF voltage if the electrical resistance and/or the increase in resistance between an ablation electrode and a measurement electrode exceeds a resistance threshold and/or has reached a minimum increase in resistance. The invention is now intended to be explained in more detail on the basis of an exemplary embodiment. To this end, FIG. 1 shows a schematic illustration of an exemplary embodiment of the instrument according to the invention and the function thereof.

An electrosurgical instrument 100 in FIG. 1 has an elongate, cylindrical shaft 20 with two ablation electrodes 1, 2 arranged in succession on the shaft 20 in the longitudinal direction L of the shaft 20. In the present case, the ablation electrodes 1, 2 have a ring-shaped embodiment and each form a surface portion of the shaft 20, with a first ablation electrode 1 simultaneously forming the distal end of the shaft 20 and hence a tip electrode. The instrument 100 also has a first insulator 5, which electrically separates the electrically conductive ablation electrodes 1, 2 from one another. It is possible to operate the ablation electrodes 1, 2 in a bipolar manner with an RF voltage by means of an RF generator (not shown).

The instrument 100 has two measurement electrodes 3, 4, which are formed between the first insulator 5 and the ablation electrodes 1, 2. The measurement electrodes are electrically conductive and electrically insulated both from the ablation electrodes 1, 2 and from one another. The two measurement electrodes 3, 4 likewise have a ring-shaped embodiment and are arranged coaxially with the ablation electrodes 1, 2 and the shaft 20. It is possible to identify that the ablation electrodes 1, 2 and the measurement electrodes 3, 4 each have a substantially identical cross-sectional measure (diameter D) with respect to the longitudinal direction L of the shaft 20.

In order to be able to detect a forming clot K1, K2 as early as possible, the first measurement electrode 3 is arranged in the direct vicinity of the first (distal) ablation electrode 1 and the second measurement electrode 4 is arranged in the direct vicinity of the second (proximal) ablation electrode 2. Here, the distance A2 between the first ablation electrode 1 and the first measurement electrode 3 is less than the distance A1 between the first measurement electrode 3 and the second measurement electrode 4. Likewise, the distance A3 between the second ablation electrode 2 and the second measurement electrode 4 is less than the distance A1 between the first measurement electrode 3 and the second measurement electrode 4.

FIG. 1 likewise shows that the first measurement electrode 3 is arranged immediately adjacent to the first ablation electrode 1, i.e., in particular, only an electrically insulating section in the form of a second insulator 5′ and no further electrodes are arranged between the first measurement electrode 3 and the ablation electrode 1. The second measurement electrode 4 and the second ablation electrode 2 are also arranged immediately adjacent to one another, separated from one another by a small distance by a narrow third insulator 5″. That is to say, the first measurement electrode 3 is delimited by the second insulator 5′ in the distal direction and by the insulator 5 in the proximal direction. By contrast, the second measurement electrode 4 is delimited by the insulator 5 in the distal direction and by the third insulator 5″ in the proximal direction.

Furthermore, the width B of the measurement electrodes 3, 4 measured in the longitudinal direction L of the shaft is less than a quarter of the cross-sectional measure (diameter D) thereof measured across the longitudinal direction. Hence, the measurement electrodes 3, 4 have a substantially smaller area than the ablation electrodes 1, 2.

The following text discusses the function of the instrument 100 during ablation operation in more detail. To this end, only the ohmic resistance R measured across the two ablation electrodes 1, 2 is plotted in FIG. 1i). Here, an “ohmic resistance” is understood to mean the magnitude of the electrical resistance.

The measurement electrodes 3, 4 are not considered in FIG. 1 i). During ablation Operation—where a bipolar RF voltage is applied to the instrument 100 and the latter is arranged in a biological tissue 300—a clot K1, K2 spreads, typically starting at an edge between the insulator 5 and the corresponding ablation electrode 1, 2. This spread is accompanied by an increase in the resistance R of the tissue 300, which is plotted over time t in FIG. 1i). However, in practical terms, only a summed electrical resistance of the tissue 300 over the total distance A1+A2+A3 between the two ablation electrodes 1, 2 is measured between the two ablation electrodes 1, 2. Thus, the clots K1, K2 can already adhere to the corresponding ablation electrode 1, 2 in the region of the edge between the insulator 5 and the corresponding ablation electrode 1, 2, without the resistance R increasing significantly.

FIG. 1ii) now shows the resistance measured across the first ablation electrode 1 and the first measurement electrode 3 for the same ablation situation which also underlies FIG. 1i). It is possible to identify that the ohmic resistance R—this time measured across the first ablation electrode 1 and the first measurement electrode 3—increases significantly more quickly than the resistance R in FIG. 1ii). This is because the resistance R measured across the first ablation electrode 1 and the first measurement electrode 3 merely relates to a tissue portion with a significantly smaller volume. FIG. 1ii) likewise plots the speed dR/dt with which the resistance R of the clot K1 increases within the scope of coagulation.

The resistance R and/or the increase in resistance over time dR/dt across the second ablation electrode 2 and the second measurement electrode 4 can also be measured analogously to FIG. 1ii). Here, the resistance R is the measured ohmic resistance. It is likewise conceivable to measure an impedance between the two ablation electrodes.

During ablation operation, a bipolar RF voltage, e.g. 500 V, is initially applied to the two ablation electrodes 1, 2 of the instrument 100. Furthermore, a resistance R and an increase in resistance dR/dt between the first ablation electrode 1 and the first measurement electrode 3, and between the second ablation electrode 2 and the second measurement electrode 4, are measured. If the resistance R exceeds a resistance threshold and/or if the increase in resistance dR/dt exceeds a minimum increase in resistance—which may be the case, for example, in the case of clots K1, K2 being present—the bipolar RF voltage applied between the ablation electrodes 1, 2 is reduced, for example to 200 V.

1. A bipolar electrosurgical instrument for ablating and/or coagulating biological tissue, comprising an elongate shaft and comprising two ablation electrodes, which are arranged in succession on the shaft in the longitudinal direction the shaft, form a surface portion of the shaft, are electrically conductive, and are electrically separated from one another by an insulator, wherein the instrument has at least one measurement electrode, which is electrically insulated from the ablation electrodes arranged between the two ablation electrodes in the immediate surroundings of one of the two ablation electrodes.

2. The instrument as claimed in claim 1, wherein the instrument has two measurement electrodes which are formed between the insulator and the ablation electrodes and are electrically insulated from the ablation electrodes.

3. The instrument as claimed in claim 1, wherein the ablation electrode and the measurement electrodes each have a ring-shaped embodiment.

4. The instrument as claimed in claim 1, wherein a first measurement electrodes is arranged in the direct vicinity of a first ablation electrode and/or a second measurement electrodes is arranged in the direct vicinity of a second ablation electrode.

5. The instrument as claimed in claim 1, wherein the measurement electrodes are arranged coaxially with respect to the shaft.

6. The instrument as claimed in claim 1, wherein, in relation to the longitudinal direction of the shaft, the ablation electrodes each have a substantially identical cross-sectional measure.

7. The instrument as claimed in claim 1, wherein the width of the measurement electrodes is less than a quarter of the diameter thereof.

8. The instrument as claimed in claim 1, wherein the area of the measurement electrodes is at most one tenth of the area of the ablation electrodes.

9. The instrument as claimed in claim 1, wherein the distance between a respective ablation electrode and the immediately adjacent measurement electrode is substantially smaller.

10. A method for operating an electrosurgical instrument as claimed in, claim 1, comprising the following steps:
applying a bipolar RF voltage to the ablation electrodes,
determining a resistance and/or an increase in resistance between an ablation electrode and an immediately adjacent measurement electrode,
reducing the bipolar RF voltage if the resistance and/or the increase in resistance exceeds a resistance threshold and/or has reached a minimum increase in resistance.
